(19) United States
(12) Patent Application Publication
(10) Pub. No.: US 2019/0321363 A1
(43) Pub. Date: Oct. 24, 2019

(54) PROCESS FOR THE PREPARATION OF ELAGOLIX SODIUM AND ITS POLYMORPH

(71) Applicant: DR. REDDY'S LABORATORIES LIMITED, Hyderabad (IN)

(72) Inventors: Subba Reddy PEDDIREDDY, Hyderabad (IN); Sunil Kumar Allam, Hyderabad (IN); Mohan Kumar KOTTUR, Hyderabad (IN); Srinivas ORUGANTI, Hyderabad (IN); Bhaskar KANDAGATLA, Hyderabad (IN)

(21) Appl. No.: 16/311,058

(22) PCT Filed: Jun. 20, 2017
(86) PCT No.: PCT/IB2017/053653

§ 371 Date: Dec. 18, 2018

(30) Foreign Application Priority Data
Jun. 20, 2016 (IN) 201641021096
Jul. 5, 2016 (IN) 201641023089

(51) Int. Cl. A61K 31/513; A61K 47/10; A61K 9/14; A61K 47/32

(52) U.S. Cl. CPC A61K 31/513; A61K 47/10; A61K 47/32; A61K 9/145; A61K 9/143

(57) ABSTRACT

The present invention provides a process for preparation of Elagolix and its intermediates, processes for preparation of amorphous Elagolix sodium and solid dispersion of Elagolix Sodium thereof.

### INTRODUCTION

The present invention provides a process for preparation of Elagolix and its intermediates, processes for preparation of amorphous Elagolix sodium and solid dispersion of Elagolix Sodium thereof.

### BACKGROUND OF THE INVENTION

Elagolix sodium is a non-peptide antagonist of the gonadotropin-releasing hormone receptor and chemically known as sodium; 4-[[(1R)-2-[5-(2-fluoro-3-methoxyphenyl)-3-[[2-fluoro-6-(trifluoromethyl)phenyl]methyl]-4-methyl-2,6-dioxopyrimidin-1-yl]-1-phenylethyl]amino] butanoate as below.

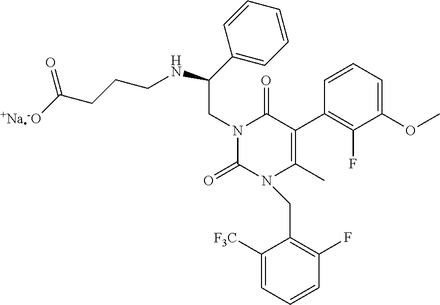

The U.S. Pat. No. 7,056,927 B2 discloses, elagolix sodium salt as a white solid and process for its preparation in Example-1; Step-1H.

The U.S. Pat. No. 8,765,948 B2 discloses a process for preparation of amorphous elagolix sodium by spray drying method and solid dispersion of amorphous elagolix sodium with a polymer.

The discovery of further solid forms of an active pharmaceutical ingredient (API) can offer an opportunity to improve the performance profile of a pharmaceutical composition comprising the said API.

Processability of the API during manufacture of the pharmaceutical composition and characteristics of the finished dosage form, such as storage stability under difficult environmental conditions, such as high relative humidity and/or high temperature, can still be improved or optimized. The presence of the high energy form of the API in a pharmaceutical composition (amorphous form) usually improves the dissolution rate.

An object of the present invention is to provide a pharmaceutical composition comprising Elagolix sodium in a solid form, wherein the physicochemical stability and the dissolution characteristics of the solid form is improved, and wherein Elagolix sodium is rendered more suitable for use in a pharmaceutical composition.

The U.S. Pat. No. 7,056,927 B2 discloses a process for preparation of elagolix sodium salt in Example-1 as given in below scheme-I.

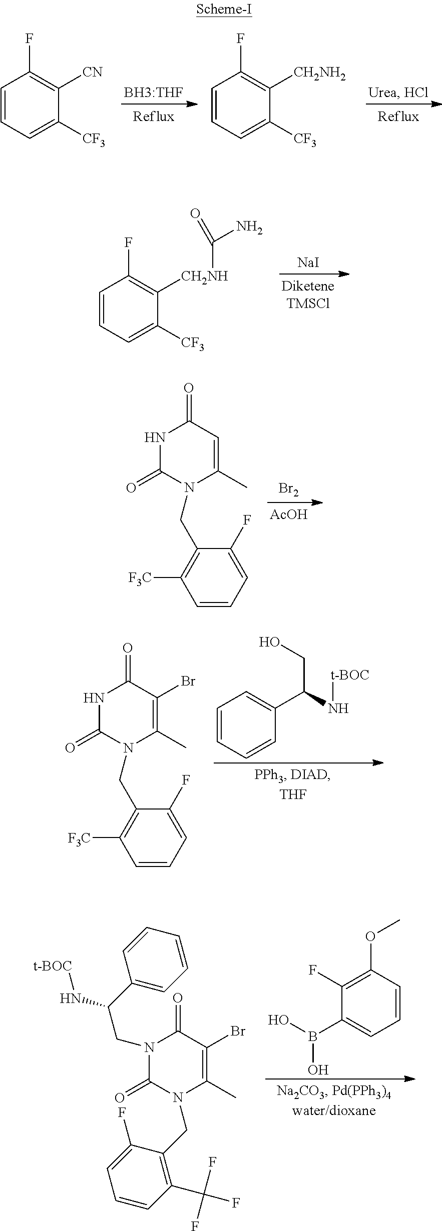

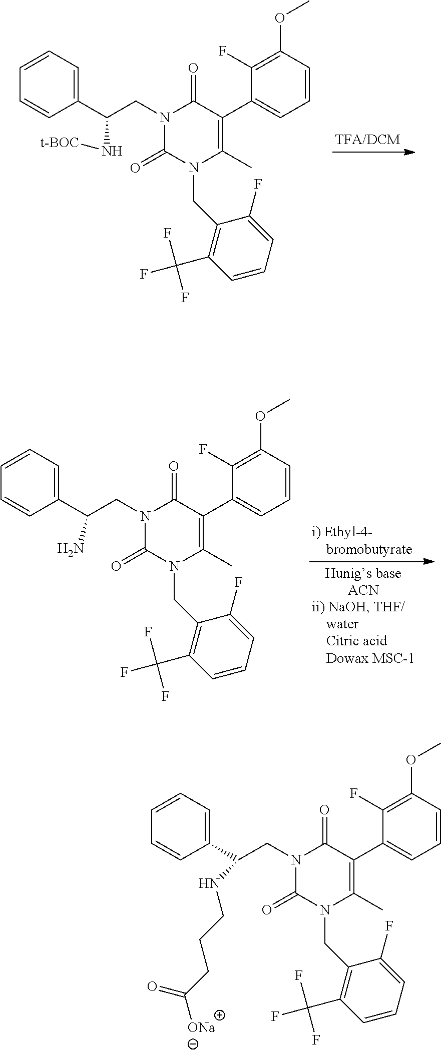

The U.S. Pat. No. 8,765,948 B2 describes a process for preparation of elagolix sodium in example-1 and 4 as given below scheme-II:

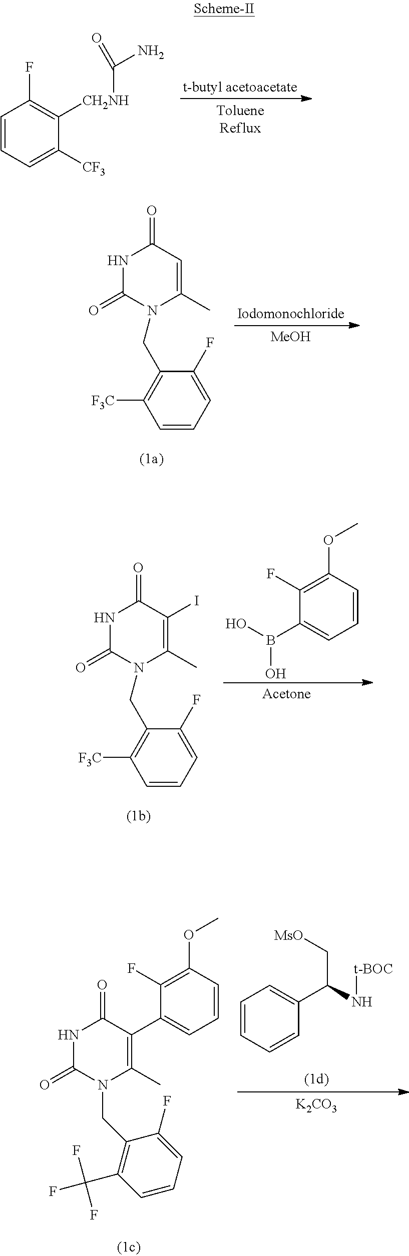

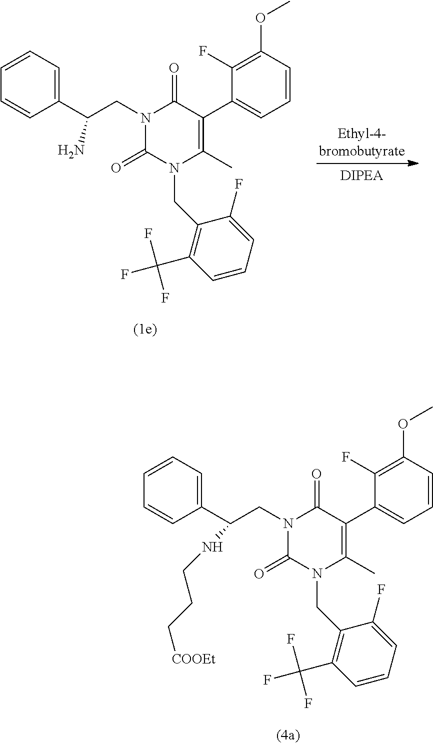 [SMILES: COc1cccc(-c2c(C)n(Cc3c(F)cccc3C(F)(F)F)c(=O)n(C[C@H](N)c3ccccc3)c2=O)c1F ; CCOC(=O)CCCN[C@@H](Cn1c(=O)c(-c2cccc(OC)c2F)c(C)n(Cc2c(F)cccc2C(F)(F)F)c1=O)c1ccccc1]

Further, the U.S. Pat. No. 8,765,948 B2 discloses an alternate process for the preparation of compound of formula (1e) as mentioned below scheme-III.

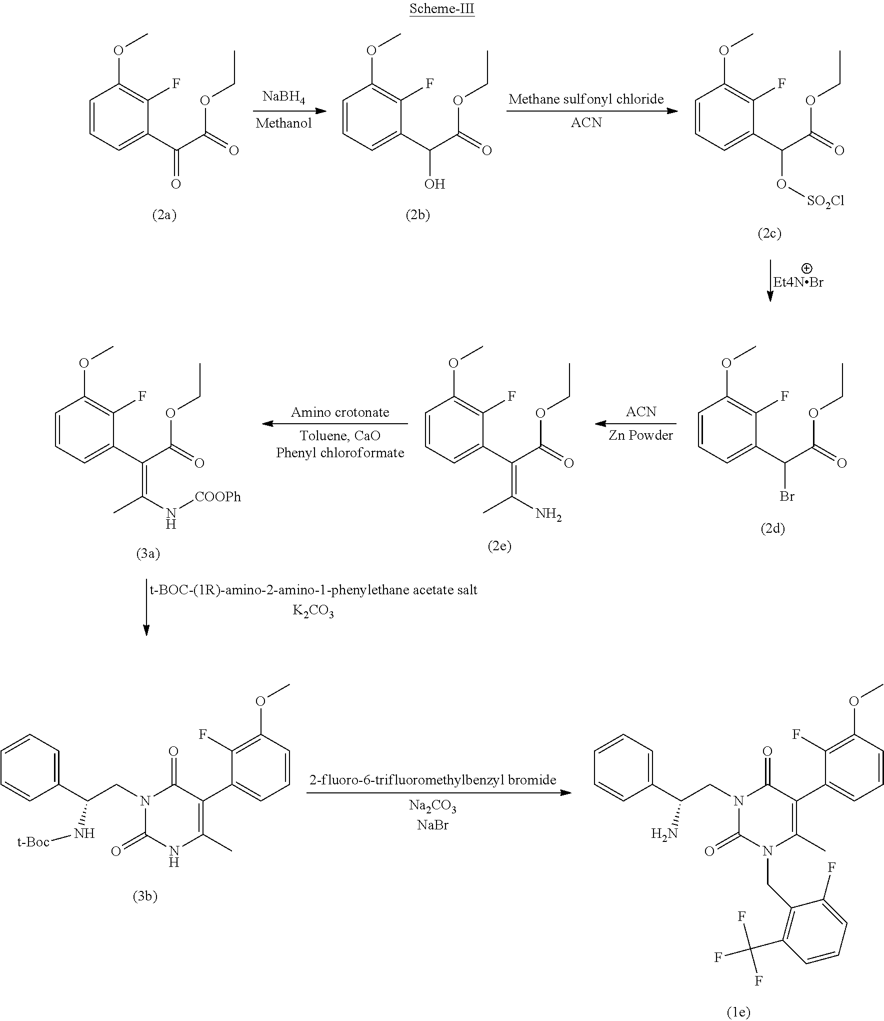

The present invention provides an improved and commercially viable process for the preparation of elagolix and intermediates thereof.

### SUMMARY

In an aspect, the present invention provides process for the preparation of amorphous Elagolix sodium, amorphous solid dispersion of Elagolix sodium and solid dispersion comprising amorphous Elagolix sodium and one or more pharmaceutically acceptable carriers.

In a second aspect, the present invention provides a process for the preparation of amorphous Elagolix sodium, comprising the steps;

a) providing a solution of Elagolix sodium in a solvent or mixture of solvents; and

b) isolating amorphous Elagolix sodium.

In a third aspect, the present invention provides a process for the preparation of amorphous Elagolix sodium, comprising the step;

a) ball milling Elagolix sodium under suitable milling conditions.

In a fourth aspect, the present invention provides a process for the preparation of amorphous solid dispersion of Elagolix sodium comprising the steps:

a) providing a solution comprising Elagolix sodium and one or more pharmaceutically acceptable carriers in a solvent,

b) removing the solvent from the solution obtained in step (a) and,

c) recovering amorphous solid dispersion of Elagolix sodium.

In a fifth aspect, the present invention provides a process for the preparation of solid dispersion comprising amorphous Elagolix sodium and one or more pharmaceutically acceptable carriers, comprising the steps:

a) providing a solution comprising Elagolix sodium and one or more pharmaceutically acceptable carriers in a solvent,

b) removing the solvent from the solution obtained in step (a) and,

c) recovering a solid dispersion comprising amorphous Elagolix sodium and one or more pharmaceutically acceptable carriers.

In a sixth aspect, the present invention provides a process for the preparation of amorphous solid dispersion of Elagolix sodium comprising the steps;

a) grinding Elagolix sodium and one or more pharmaceutically acceptable carriers and

b) recovering amorphous solid dispersion of Elagolix sodium.

In a seventh aspect, the present invention provides a process for preparation of the compound of formula (VII) which is an intermediate in the preparation process of Elagolix.

In an eighth aspect, the present invention provides a process for preparation of the compound of formula (III) which is an intermediate in the preparation process of Elagolix.

In a ninth aspect, the present invention provides a process for preparation of Elagolix or its pharmaceutically acceptable salt.

### BRIEF DESCRIPTION OF THE DRAWINGS

FIG. 1 illustrates the PXRD pattern of amorphous Elagolix sodium, obtained by the procedure of Example 1.

FIG. 2 illustrates the PXRD pattern of amorphous Elagolix sodium, obtained by the procedure of Example 2.

FIG. 3 illustrates the PXRD pattern of amorphous Elagolix sodium, obtained by the procedure of Example 3.

FIG. 4 illustrates the PXRD pattern of amorphous Elagolix sodium, obtained by the procedure of Example 4.

FIG. 5 illustrates the PXRD pattern of amorphous Elagolix sodium, obtained by the procedure of Example 5.

FIG. 6 illustrates the PXRD pattern of amorphous solid dispersion of Elagolix sodium, obtained by the procedure of Example 6.

FIG. 7 illustrates the PXRD pattern of amorphous solid dispersion of Elagolix sodium, obtained by the procedure of Example 7.

FIG. 8 illustrates the PXRD pattern of solid dispersion comprising amorphous Elagolix sodium with PEG-8000 obtained by the procedure of Example 8.

FIG. 8(i) illustrates the PXRD pattern of PEG-8000 used in the procedure of Example 8.

FIG. 9 illustrates the PXRD pattern of amorphous solid dispersion of Elagolix sodium, obtained by the procedure of Example 9.

FIG. 10 illustrates the PXRD pattern of amorphous solid dispersion of Elagolix sodium, obtained by the procedure of Example 10.

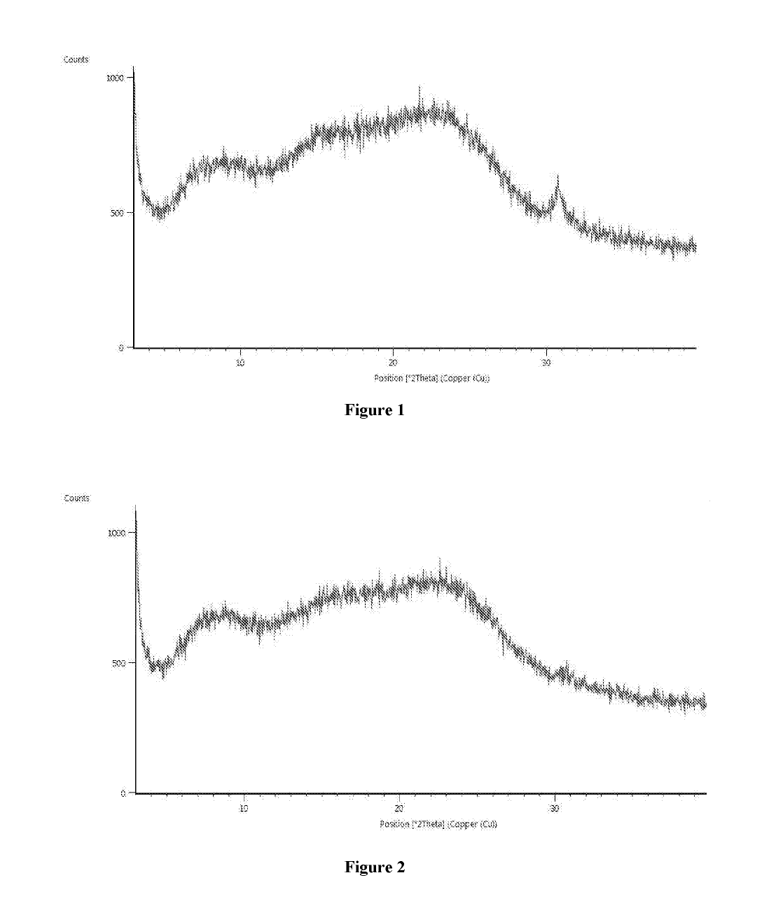

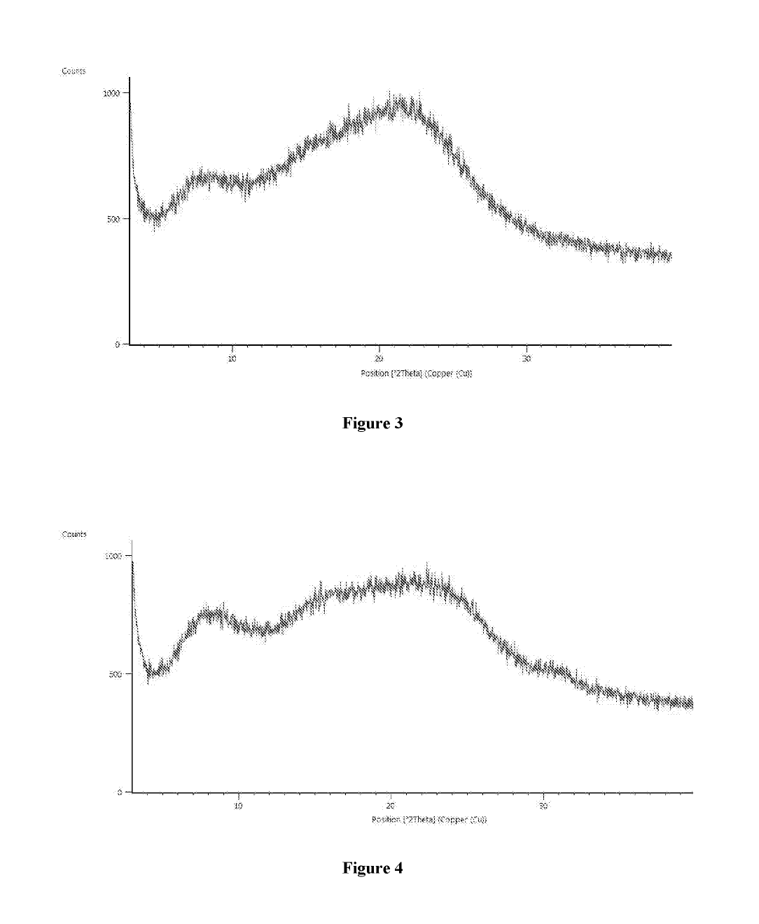

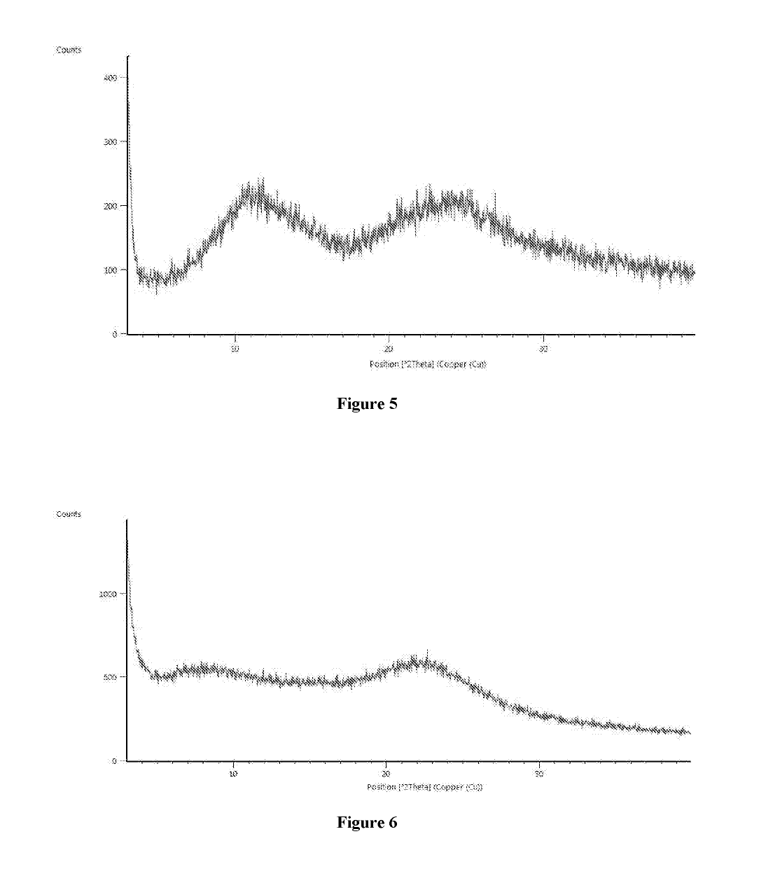

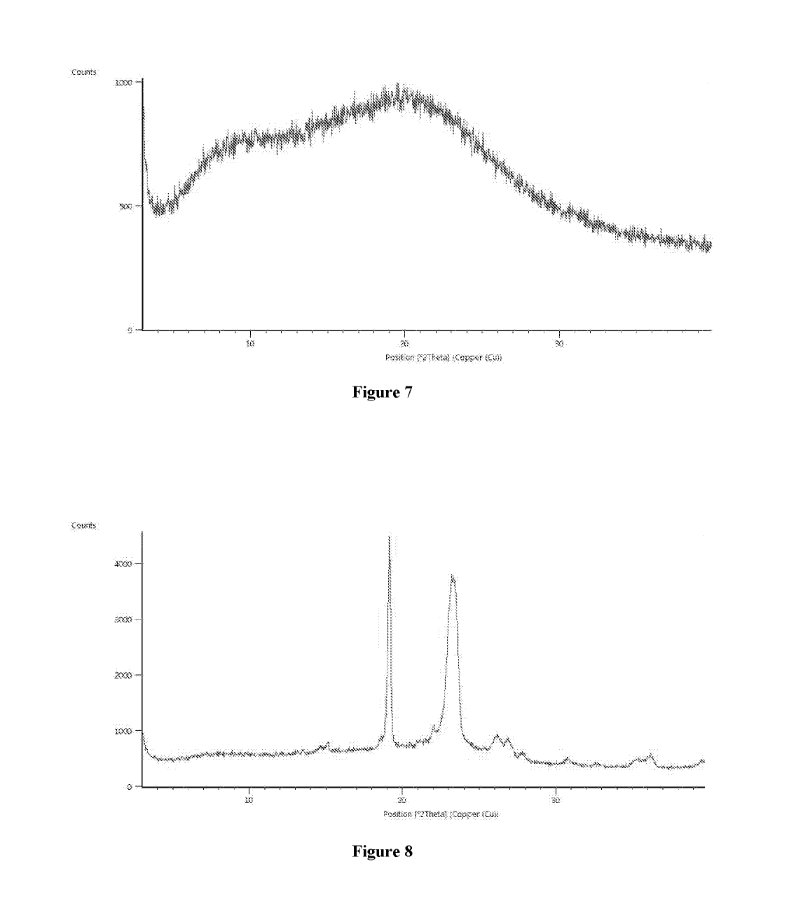

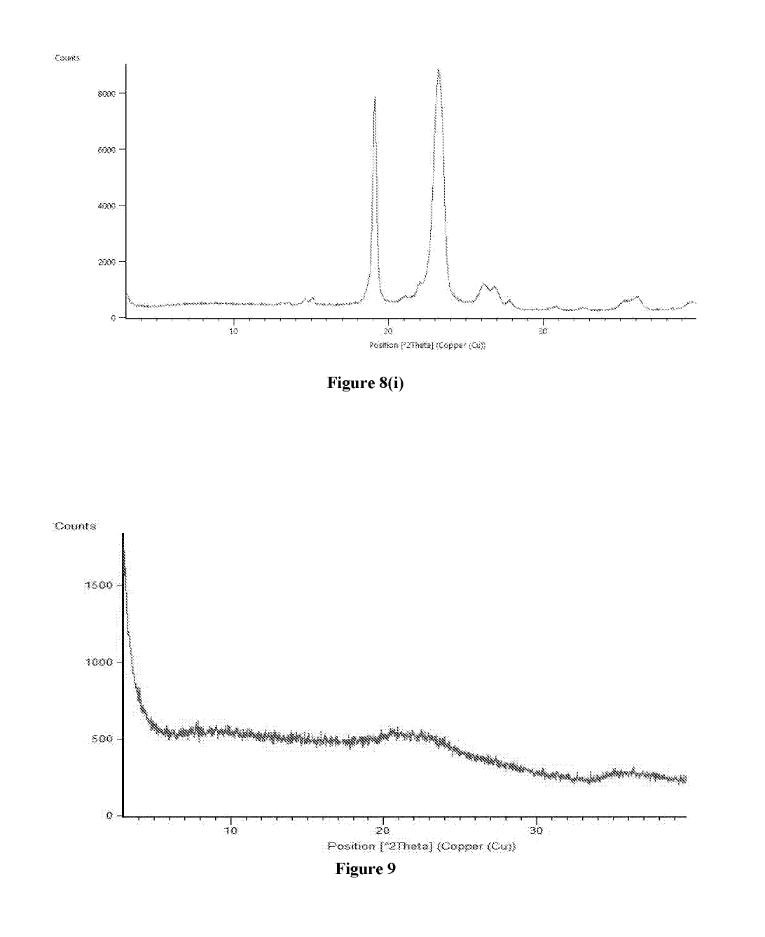

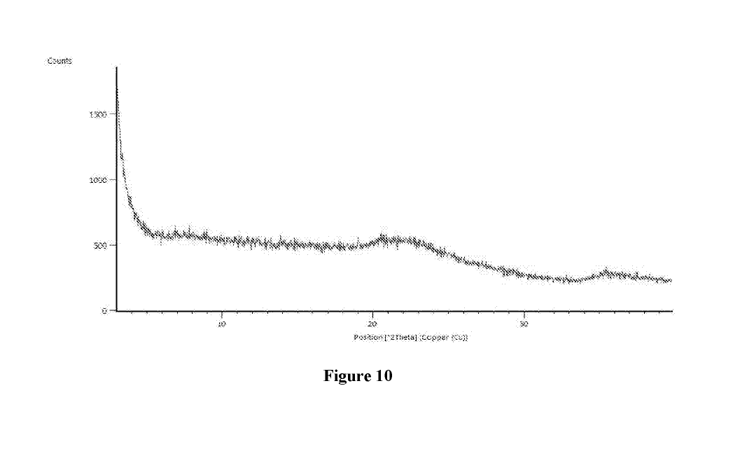

### DETAILED DESCRIPTION

In an aspect, the present invention provides process for the preparation of amorphous Elagolix sodium, amorphous solid dispersion of Elagolix sodium and solid dispersion comprising amorphous Elagolix sodium and one or more pharmaceutically acceptable carriers.

In a second aspect, the present invention provides a process for the preparation of amorphous Elagolix sodium, comprising the steps;

a) providing a solution of Elagolix sodium in a solvent or mixture of solvents and,

b) isolating amorphous Elagolix sodium.

Providing a solution of Elagolix sodium in step a) includes:

i) direct use of a reaction mixture containing Elagolix sodium that is obtained in the course of its synthesis; or

ii) dissolving Elagolix sodium in a solvent.

Any physical form of Elagolix sodium may be utilized for providing the solution of Elagolix sodium in step a). The dissolution temperatures may range from about 0° C. to about the reflux temperature of the solvent, or less than about 70° C., less than about 50° C., less than about 40° C., less than about 30° C., less than about 20° C., less than about 10° C., or any other suitable temperatures, as long as a clear solution of Elagolix sodium is obtained without affecting its quality. The solution may optionally be treated with carbon, flux-calcined diatomaceous earth (Hyflow) or any other suitable material to remove color, insoluble materials, improve clarity of the solution, and/or remove impurities adsorbable on such material. Optionally, the solution obtained above may be filtered to remove any insoluble particles. The insoluble particles may be removed suitably by filtration, centrifugation, decantation, or any other suitable techniques under pressure or under reduced pressure. The solution may be filtered by passing through paper, glass fiber, cloth or other membrane material, or a bed of a clarifying agent such as Celite® or Hyflow. Depending upon the equipment used and the concentration and temperature of the solution, the filtration apparatus may need to be preheated to avoid premature crystallization.

In embodiments, Elagolix sodium can be dissolved in the following solvents. Examples of the solvents comprises alcohols, such as methanol, ethanol, 1-propanol, 2-propanol (isopropyl alcohol), 2-methoxyethanol, 1-butanol, 2-butanol, iso-butyl alcohol, t-butyl alcohol, 2-ethoxyethanol, diethylene glycol, 1-, 2-, or 3-pentanol, neo-pentyl alcohol, t-pentyl alcohol, diethylene glycol monomethyl ether, cyclohexanol, glycerol, or C1-C6 alcohols and the like; nitriles, such as acetonitrile or propionitrile; amides, such as formamide, N,N-dimethylformamide, N,N-dimethylacetamide, N-methyl-2-pyrrolidone, or hexamethyl phosphoric triamide and the like; sulfoxides, such as dimethylsulfoxide and the like; or any mixtures of two or more solvents thereof. In a more preferred embodiment solvents such as alcohols, chlorohydrocarbons or mixture thereof are used.

The quantity of solvent used for dissolution depends on the solvent and the dissolution temperature adopted.

Step b) involves isolating amorphous Elagolix sodium from the solution obtained in step a). Isolation of amorphous Elagolix sodium in step b) may involve methods including removal of solvent, cooling, crash cooling, concentrating the mass, evaporation, flash evaporation, simple evaporation, fast solvent evaporation, rotational drying, spray drying, thin-film drying, agitated thin film drying, agitated nutsche filter drying, pressure nutsche filter drying, freeze-drying, rotary vacuum paddle dryer, adding anti-solvent or the like. Stirring or other alternate methods such as shaking, agitation, or the like, may also be employed for the isolation. The amorphous Elagolix sodium as isolated may carry some amount of occluded mother liquor and may have higher than desired levels of impurities. If desired, this amorphous form may be washed with a solvent or a mixture of solvents to wash out the impurities.

Suitable temperatures for isolation may be less than about 60° C., less than about 40° C., less than about 30° C., less than about 20° C., less than about 10° C., less than about 0° C., less than about −10° C., less than about −40° C. or any other suitable temperatures.

Optionally isolation of amorphous Elagolix sodium may also be effected by combining a suitable anti-solvent with the solution obtained in step a). Anti-solvent as used herein refers to a liquid in which Elagolix sodium is less soluble or poorly soluble. An inert anti-solvent has no adverse effect on the reaction and it can assist in the solidification or precipitation of the dissolved starting material. Suitable anti-solvents that may be used include, but are not limited to: saturated or unsaturated, linear or branched, cyclic or acyclic, C₁ to C₁₀ hydrocarbons, such as heptanes, cyclohexane, or methylcyclohexane; water; or any mixtures thereof.

The recovered solid may optionally be dried. Drying may be carried out in a tray dryer, vacuum oven, air oven, cone vacuum dryer, rotary vacuum dryer, fluidized bed dryer, spin flash dryer, flash dryer, or the like. The drying may be carried out at temperatures less than about 100° C., less than about 80° C., less than about 60° C., less than about 50° C., less than about 30° C., or any other suitable temperatures, at atmospheric pressure or under a reduced pressure, as long as the Elagolix sodium is not degraded in quality. The drying may be carried out for any desired times until the required product quality is achieved. The dried product may optionally be subjected to a size reduction procedure to produce desired particle sizes. Milling or micronization may be performed before drying, or after the completion of drying of the product. Techniques that may be used for particle size reduction include, without limitation, ball, roller or hammer milling; or jet milling; or bead milling.

In a third aspect, the present invention provides a process for the preparation of amorphous Elagolix sodium, comprising the step;

a) ball milling Elagolix sodium under suitable milling conditions.

Any solid forms, either crystalline or amorphous form of Elagolix sodium can be used to mill it with one or more pharmaceutically acceptable carriers.

In a fourth aspect, the present invention provides a process for the preparation of amorphous solid dispersion of Elagolix sodium comprising the steps:

a) providing a solution comprising Elagolix sodium and one or more pharmaceutically acceptable carriers in a solvent,

b) removing the solvent from the solution obtained in step (a) and,

c) recovering amorphous solid dispersion of Elagolix sodium.

Step a) involves providing a solution of Elagolix sodium and at least one pharmaceutically acceptable carrier in a solvent;

Step a) may involve forming a solution of Elagolix sodium and one or more pharmaceutically acceptable carriers. In embodiments, the carrier enhances stability of the amorphous solid upon removal of solvent.

Providing the solution in step a) includes:

i) direct use of a reaction mixture containing Elagolix sodium that is obtained in the course of its manufacture, if desired, after addition of one or more pharmaceutically acceptable carriers; or
ii) dissolution of Elagolix sodium in a solvent, either alone or in combination with one or more pharmaceutically acceptable carriers.

The quantity of solvent used for dissolution depends on the solvent and the dissolution temperature adopted.

Any physical form of Elagolix sodium, such as crystalline, amorphous or their mixtures may be utilized for providing a solution in step a).

Pharmaceutically acceptable carriers that may be used in step a) include, but are not limited to: diluents such as starches, pregelatinized starches, lactose, powdered celluloses, microcrystalline celluloses, dicalcium phosphate, tricalcium phosphate, mannitol, sorbitol, sugar, or the like; binders such as acacia, guar gum, tragacanth, gelatin, Neusilin® (Magnesium Alumino-metasilicate), polyvinylpyrrolidones (PVP), Polyvinylpyrrolidone K 30 (PVPK-30), Polyethylene glycol, copovidone, hydroxypropyl celluloses, hydroxypropyl methyl celluloses, pregelatinized starches, or the like; disintegrants such as starches, sodium starch glycolate, pregelatinized starches, crospovidones, croscarmellose sodium, colloidal silicon dioxide, or the like; lubricants such as stearic acid, magnesium stearate, zinc stearate, or the like; glidants such as colloidal silicon dioxide or the like; solubility or wetting enhancers such as anionic or cationic or neutral surfactants; complex forming agents such as various grades of cyclodextrins or resins; release rate controlling agents such as hydroxypropyl celluloses (HPC), hydroxymethyl celluloses, hydroxyethylcellulose, hydroxyethylmethylcellulose (HEMC), carboxymethylcellulose (CMC), carboxymethylhydroxyethylcellulose (CMHEC), hydroxyethylcarboxymethylcellulose (HECMC), sodium carboxymethylcellulose, cellulose acetate phthalate (CAP), hydroxypropyl methylcelluloses (HPMC), hydroxypropylmethylcellulose acetate (HPMCA), hydroxypropylmethylcellulose phthalate (HPMCP), hydroxypropyl methyl cellulose acetate succinate (HPMCAS), Low-Substituted Hydroxypropyl Cellulose (HPC-L), ethylcelluloses, methylcelluloses, propylcellulose, hydroxyethyl methyl cellulose, hydroxyethyl cellulose acetate, hydroxyethyl ethyl cellulose, Syloid®, various grades of methyl methacrylates, poly(meth)acrylates (EUDRAGIT®), waxes, Soluplus® or the like. Other pharmaceutically acceptable excipients that are of use include, but are not limited to, film formers, plasticizers, colorants, flavoring agents, sweeteners, viscosity enhancers, preservatives, antioxidants, or the like.

Elagolix sodium and the pharmaceutically acceptable carriers may be dissolved either in the same solvent or they may be dissolved in different solvents and then combined to form a mixture. In embodiments, the solid dispersion described herein comprises amorphous Elagolix sodium and the carrier present in weight ratios ranging from about 5:95 to about 95:5. An example of a ratio is about 50:50. In some embodiments, the solid dispersion described herein comprises one or more pharmaceutically acceptable excipients, preferably two excipients.

The dissolution temperatures may range from about 0° C. to about the reflux temperature of the solvent, or less than about 60° C., less than about 50° C., less than about 40° C., less than about 30° C., less than about 20° C., less than about 10° C., or any other suitable temperatures, as long as a clear solution of Elagolix sodium is obtained without affecting its quality. The solution may optionally be treated with carbon, flux-calcined diatomaceous earth (Hyflow) or any other suitable material to remove color, insoluble materials, improve clarity of the solution, and/or remove impurities adsorbable on such material. Optionally, the solution obtained above may be filtered to remove any insoluble particles. The insoluble particles may be removed suitably by filtration, centrifugation, decantation, or any other suitable techniques under pressure or under reduced pressure. The solution may be filtered by passing through paper, glass fiber, cloth or other membrane material, or a bed of a clarifying agent such as Celite® or Hyflow. Depending upon the equipment used and the concentration and temperature of the solution, the filtration apparatus may need to be preheated to avoid premature crystallization.

The solvents that may be used in step a) include but are not limited to: alcohols, such as methanol, ethanol, 2-nitroethanol, 2-fluoroethanol, ethylene glycol, 1-propanol, 2-propanol (isopropyl alcohol), 2-methoxyethanol, 1-butanol, 2-butanol, iso-butyl alcohol, t-butyl alcohol, 2-ethoxyethanol, neo-pentyl alcohol, t-pentyl alcohol, diethylene glycol monomethyl ether, cyclohexanol, or C₁-C₆ alcohols and the like; nitriles, such as acetonitrile or propionitrile; amides, such as formamide, N,N-dimethylformamide, N,N-dimethylacetamide, N-methyl-2-pyrrolidone, or hexamethyl phosphoric triamide and the like; sulfoxides, such as dimethylsulfoxide and the like;

Step b) involves removal of the solvent from the solution comprising Elagolix sodium and one or more pharmaceutically acceptable carriers. The solvent can be removed using the techniques such as evaporation, spray drying and other conventional techniques.

Step c) involves recovering the amorphous solid dispersion comprising Elagolix sodium and one or more pharmaceutically acceptable carriers.

A solid amorphous dispersion comprising Elagolix sodium and one or more pharmaceutically acceptable carriers may be isolated from a solution comprising Elagolix sodium and one or more pharmaceutically acceptable carriers in a solvent by using the conventional methods. The methods includes but not limited to cooling, crash cooling, concentrating the mass, evaporation, flash evaporation, simple evaporation, fast solvent evaporation, rotational drying, spray drying, thin-film drying, agitated thin-film drying, agitated nutsche filter drying, pressure nutsche filter drying, freeze-drying or the like. Stirring or other alternate methods such as shaking, agitation, or the like, may also be employed for the isolation. The amorphous Elagolix sodium as isolated may carry some amount of occluded mother liquor and may have higher than desired levels of impurities. If desired, this amorphous form may be washed with a solvent or a mixture of solvents to wash out the impurities.

The recovered solid dispersion may optionally be dried. Drying may be carried out in a tray dryer, vacuum oven, air oven, cone vacuum dryer, rotary vacuum dryer, fluidized bed dryer, spin flash dryer, flash dryer, or the like. The drying may be carried out at temperatures less than about 100° C., less than about 80° C., less than about 60° C., less than about 50° C., less than about 30° C., or any other suitable temperatures, at atmospheric pressure or under a reduced pressure, as long as the Elagolix sodium is not degraded in quality. The drying may be carried out for any desired times until the required product quality is achieved. The dried product may optionally be subjected to a size reduction procedure to produce desired particle sizes. Milling or micronization may be performed before drying, or after the completion of drying of the product. Techniques that may be used for particle size reduction include, without limitation, ball, roller or hammer milling; or jet milling; or bead milling.

In a fifth aspect, the present invention provides a process for the preparation of solid dispersion comprising amorphous Elagolix sodium and one or more pharmaceutically acceptable carriers, comprising the steps:

a) providing a solution comprising Elagolix sodium and one or more pharmaceutically acceptable carriers in a solvent,

b) removing the solvent from the solution obtained in step (a) and,

c) recovering a solid dispersion comprising amorphous Elagolix sodium and one or more pharmaceutically acceptable carriers.

Step a) involves providing a solution of Elagolix sodium and at least one pharmaceutically acceptable carrier in a solvent;

Step a) may involve forming a solution of Elagolix sodium and one or more pharmaceutically acceptable carriers. In embodiments, the carrier enhances stability of the amorphous solid upon removal of solvent.

Providing the solution in step a) includes:
i) direct use of a reaction mixture containing Elagolix sodium that is obtained in the course of its manufacture, if desired, after addition of one or more pharmaceutically acceptable carriers; or
ii) dissolution of Elagolix sodium in a solvent, either alone or in combination with one or more pharmaceutically acceptable carriers.

The quantity of solvent used for dissolution depends on the solvent and the dissolution temperature adopted. The concentration of Elagolix sodium in the solution may generally range from about 0.1 to about 10 g/ml in the solvent.

Any physical form of Elagolix sodium, such as crystalline, amorphous or their mixtures may be utilized for providing a solution in step a).

Pharmaceutically acceptable carriers that may be used in step a) include, but are not limited to: diluents such as starches, pregelatinized starches, lactose, powdered celluloses, microcrystalline celluloses, dicalcium phosphate, tricalcium phosphate, mannitol, sorbitol, sugar, or the like; binders such as acacia, guar gum, tragacanth, gelatin, Neusilin® (Magnesium Alumino-metasilicate), polyvinylpyrrolidones (PVP), Polyvinylpyrrolidone K 30 (PVPK-30), Polyethylene glycol, copovidone, hydroxypropyl celluloses, hydroxypropyl methyl celluloses, pregelatinized starches, or the like; disintegrants such as starches, sodium starch glycolate, pregelatinized starches, crospovidones, croscarmellose sodium, colloidal silicon dioxide, or the like; lubricants such as stearic acid, magnesium stearate, zinc stearate, or the like; glidants such as colloidal silicon dioxide or the like; solubility or wetting enhancers such as anionic or cationic or neutral surfactants; complex forming agents such as various grades of cyclodextrins or resins; release rate controlling agents such as hydroxypropyl celluloses (HPC), hydroxymethyl celluloses, hydroxyethylcellulose, hydroxyethylmethylcellulose (HEMC), carboxymethylcellulose (CMC), carboxymethylhydroxyethylcellulose (CMHEC), hydroxyethylcarboxymethylcellulose (HECMC), sodium carboxymethylcellulose, cellulose acetate phthalate (CAP), hydroxypropyl methylcelluloses (HPMC), hydroxypropylmethylcellulose acetate (HPMCA), hydroxypropylmethylcellulose phthalate (HPMCP), hydroxypropyl methyl cellulose acetate succinate (HPMCAS), Low-Substituted Hydroxypropyl Cellulose (HPC-L), ethylcelluloses, methylcelluloses, propylcellulose, hydroxyethyl methyl cellulose, hydroxyethyl cellulose acetate, hydroxyethyl ethyl cellulose, Syloid®, various grades of methyl methacrylates, poly(meth)acrylates (EUDRAGIT®), waxes, Soluplus® or the like. Other pharmaceutically acceptable excipients that are of use include, but are not limited to, film formers, plasticizers, colorants, flavoring agents, sweeteners, viscosity enhancers, preservatives, antioxidants, or the like.

Elagolix sodium and the pharmaceutically acceptable carriers may be dissolved either in the same solvent or they may be dissolved in different solvents and then combined to form a mixture. In embodiments, the solid dispersion described herein comprises amorphous Elagolix sodium and the carrier present in weight ratios ranging from about 5:95 to about 95:5 by weight. An example of a ratio is about 50:50 by weight. In some embodiments, the solid dispersion described herein comprises one or more pharmaceutically acceptable excipients, preferably two excipients.

The dissolution temperatures may range from about 0° C. to about the reflux temperature of the solvent, or less than about 45° C., less than about 45° C., less than about 40° C., less than about 30° C., less than about 20° C., less than about 10° C., or any other suitable temperatures, as long as a clear solution of Elagolix sodium is obtained without affecting its quality. The solution may optionally be treated with carbon, flux-calcined diatomaceous earth (Hyflow) or any other suitable material to remove color, insoluble materials, improve clarity of the solution, and/or remove impurities adsorbable on such material. Optionally, the solution obtained above may be filtered to remove any insoluble particles. The insoluble particles may be removed suitably by filtration, centrifugation, decantation, or any other suitable techniques under pressure or under reduced pressure. The solution may be filtered by passing through paper, glass fiber, cloth or other membrane material, or a bed of a clarifying agent such as Celite® or Hyflow. Depending upon the equipment used and the concentration and temperature of the solution, the filtration apparatus may need to be preheated to avoid premature crystallization.

The solvents that may be used in step a) include but are not limited to: alcohols, such as methanol, ethanol, 2-nitroethanol, 2-fluoroethanol, ethylene glycol, 1-propanol, 2-propanol (isopropyl alcohol), 2-methoxyethanol, 1-butanol, 2-butanol, iso-butyl alcohol, t-butyl alcohol, 2-ethoxyethanol, neo-pentyl alcohol, t-pentyl alcohol, diethylene glycol monomethyl ether, cyclohexanol, or C1-C6 alcohols and the like; nitriles, such as acetonitrile or propionitrile; amides, such as formamide, N,N-dimethylformamide, N,N-dimethylacetamide, N-methyl-2-pyrrolidone, or hexamethyl phosphoric triamide and the like; sulfoxides, such as dimethylsulfoxide and the like;

Step b) involves removal of the solvent from the solution comprising Elagolix sodium and one or more pharmaceutically acceptable carriers. The solvent can be removed using the techniques such as evaporation, spray drying and other conventional techniques.

Step c) involves recovering the solid dispersion comprising amorphous Elagolix sodium and one or more pharmaceutically acceptable carriers.

A solid dispersion comprising amorphous Elagolix sodium and one or more pharmaceutically acceptable carriers may be isolated from a solution comprising Elagolix sodium and one or more pharmaceutically acceptable carriers in a solvent by using the conventional methods. The methods includes but not limited to cooling, crash cooling, concentrating the mass, evaporation, flash evaporation, simple evaporation, fast solvent evaporation, rotational drying, spray drying, thin-film drying, agitated thin-film drying, agitated nutsche filter drying, pressure nutsche filter drying, freeze-drying or the like. Stirring or other alternate methods such as shaking, agitation, or the like, may also be employed for the isolation. The amorphous Elagolix sodium as isolated may carry some amount of occluded mother liquor and may have higher than desired levels of impurities. If desired, this amorphous form may be washed with a solvent or a mixture of solvents to wash out the impurities.

The recovered solid dispersion may optionally be dried. Drying may be carried out in a tray dryer, vacuum oven, air oven, cone vacuum dryer, rotary vacuum dryer, fluidized bed dryer, spin flash dryer, flash dryer, or the like. The drying may be carried out at temperatures less than about 100° C., less than about 80° C., less than about 60° C., less than about 50° C., less than about 30° C., or any other suitable temperatures, at atmospheric pressure or under a reduced pressure, as long as the Elagolix sodium is not degraded in quality. The drying may be carried out for any desired times until the required product quality is achieved. The dried product may optionally be subjected to a size reduction procedure to produce desired particle sizes. Milling or micronization may be performed before drying, or after the completion of drying of the product. Techniques that may be used for particle size reduction include, without limitation, ball, roller or hammer milling; or jet milling; or bead milling.

In a sixth aspect, the present invention further provides a process for preparation of solid dispersion comprising amorphous Elagolix sodium and one or more pharmaceutically acceptable carriers, comprising the steps of:
a) grinding Elagolix sodium and one or more pharmaceutically acceptable carriers and
b) recovering a solid dispersion comprising amorphous Elagolix sodium and one or more pharmaceutically acceptable carriers.

Any solid forms, either crystalline or amorphous form of Elagolix sodium can be used to grind it with one or more pharmaceutically acceptable carriers.

The present invention also provides a pharmaceutical composition comprising the solid dispersion as described above.

The pharmaceutical composition of the present invention may be formulated in accordance with conventional methods, and may be prepared in the form of oral formulations such as tablets, pills, powders, capsules, syrups, emulsions, micro emulsions, and others, or formulation for parenteral injection, e.g., intramuscular, intravenous, or subcutaneous administration. The pharmaceutical composition of the present invention may comprise the inventive solid dispersion, and any possible carrier and excipient.

In a seventh aspect, the present invention provides a process for preparation of compound of formula (VII)

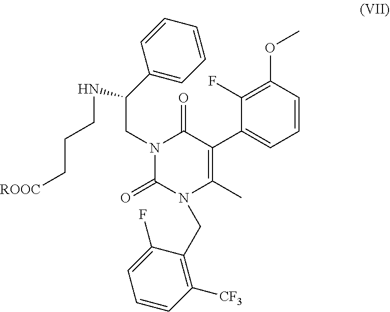

wherein R is alkyl such as methyl, ethyl, propyl, isopropyl and the like.
comprising;

a) reacting the compound of formula (II) with compound of formula (III) to obtain the compound of formula (IV)

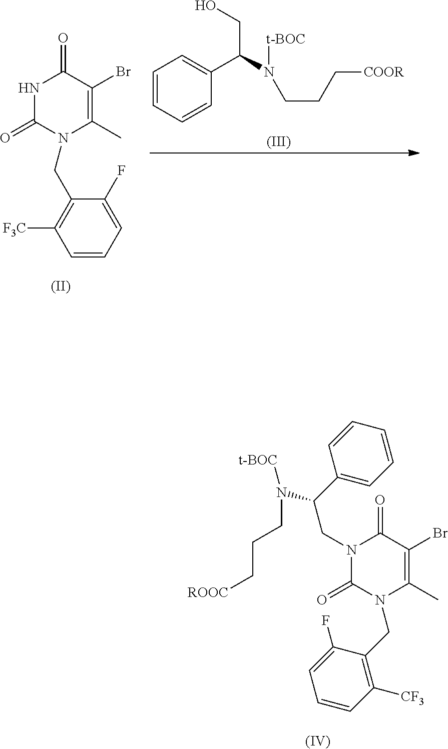

wherein t-BOC is tertiary butoxycarbonyl group; R is as described above

b) reacting the compound of formula (IV) with the compound of formula (V) to obtain the compound formula (VI), and

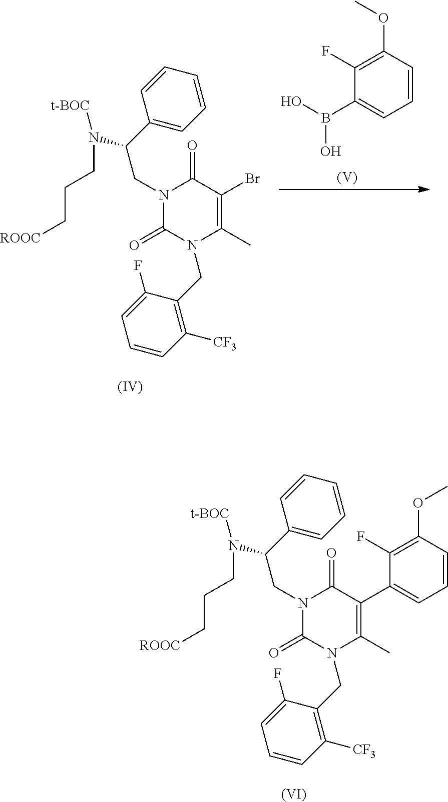

c) N-deprotection of the compound of formula (VI) to obtain the compound of formula (VII)

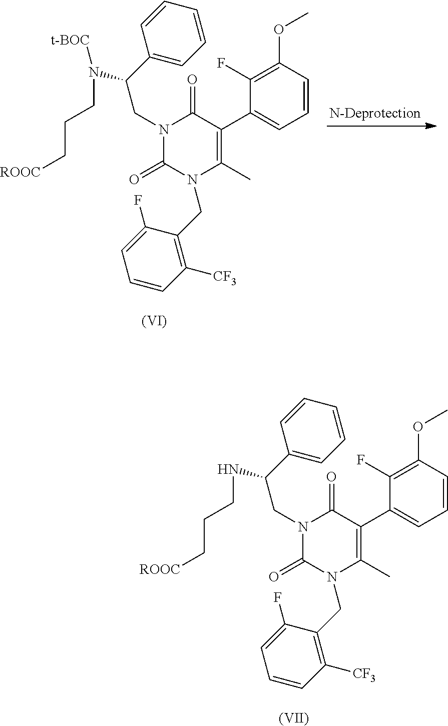

The reaction of compound of formula (II) with compound of formula (III) to obtain the compound of formula (IV) is carried in the presence of triarylphosphine such as triphenyl phosphine and the like and azodicarboxylates such as diethyl azodicarboxylate, diisopropyl azodicarboxylate and di-tert-butyl azodicarboxylate (DIAD) and the like.

The solvents used for the reaction of compound of formula (II) with compound of formula (III) include ether solvent such as anhydrous diethyl ether, dioxane, tetrahydrofuran and the like; hydrocarbon such as hexane, toluene and the like; halogenated hydrocarbon such as dichloromethane, trichloromethane and the like or the combination thereof. The reaction carried out in the temperature range of 0-60° C., particularly 20-40° C.

The present invention also includes reaction of appropriate halo, mesylate or sulfonate ester of compound of formula (III) with compound of formula (II) to obtain the compound of formula (IV).

The reaction of compound of formula (IV) with compound of formula (V) to provide the compound of formula (VI) carried out under conditions used in Suzuki coupling reaction. Specifically, the reaction involves use of palladium catalyst such as Pd(PPh₃)₄, Pd(OAc)₂ and the like. The reaction also uses inorganic base such as alkali metal carbonate comprising sodium carbonate, potassium carbonate and the like; alkali metal phosphates such as sodium phosphate, potassium phosphate and the like; alkali hydroxides such as sodium hydroxide, potassium hydroxide and the like. The solvent used in the reaction comprises ether such as diethylether, isopropyl ether, 1,4-dioxane, tetrahydrofuran and the like; ketone solvent such as acetone, methyl ethyl ketone and the like; hydrocarbon such as hexane, toluene and the like; alcohol solvent such as methanol, ethanol and the like; amide solvents such as dimethyl formamide, dimethyl acetamide and the like or the combination thereof. The reaction carried out in the temperature range of 0-120° C., specifically 30-100° C., more specifically 60-90° C.

The N-deprotection of compound of formula (VI) to obtain the compound of formula (VII) carried in the presence of acid. The acid comprises hydrochloric acid, trifluoroacetic acid, phosphoric acid and the like. The solvent used for deprotection selected from hydrocarbon such as hexane, toluene and the like; halogenated hydrocarbon such as dichloromethane, trichloromethane and the like; alcohol such as methanol, ethanol, propanol and the like; ether such as diethyl ether, isopropyl ether and the like; ester solvent such ethylacetate, isopropyl acetate and the like.

The N-deprotection reaction is carried in the temperature range of −20° C. to 60° C., specifically 10-40° C., more specifically 0-20° C.

In each stage the compounds are isolated from the reaction mixture may involve methods including removal of solvent, cooling, crash cooling, concentrating the mass, evaporation, flash evaporation, simple evaporation, fast solvent evaporation, rotational drying, spray drying, thin-film drying, agitated thin film drying, agitated nutsche filter drying, pressure nutsche filter drying, freeze-drying, rotary vacuum paddle dryer, adding anti-solvent or the like. Stirring or other alternate methods such as shaking, agitation, or the like, may also be employed for the isolation.

The present invention also covers in-situ preparation of compound of formula (VII) from the compound of formula (II) as described herein.

The present invention also covers the different order of the various N-alkylation, Suzuki coupling reaction and N-deprotection.

The seventh aspect of the present invention is depicted below scheme-IV.

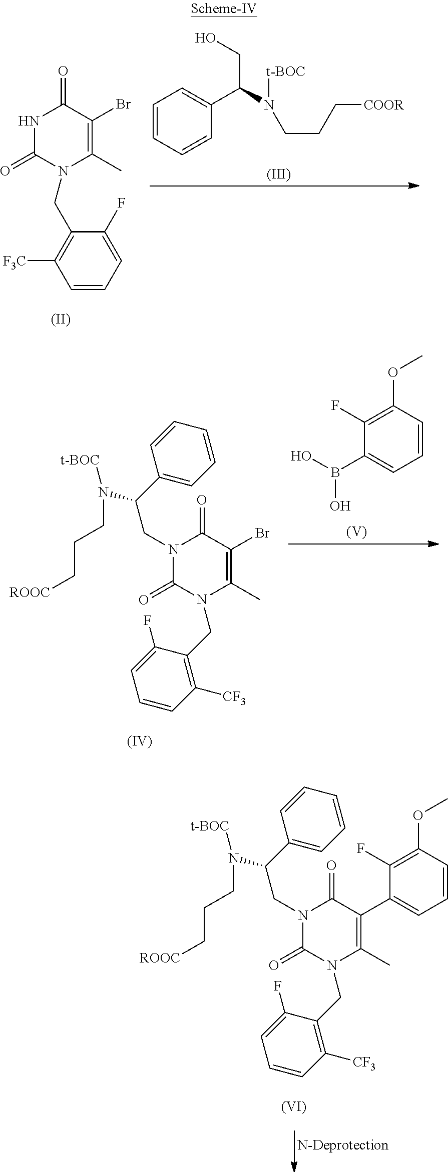

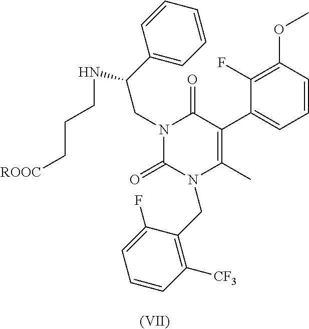

In an eighth aspect, the present invention provides a process for preparation of compound of formula (III)

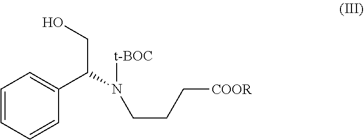

wherein R is alkyl such as methyl, ethyl, propyl and the like
comprising;
a) reacting R-(−)-2-phenylglycinol with 4-halao butyric acid alkyl ester to obtain the compound of formula (IIIa)

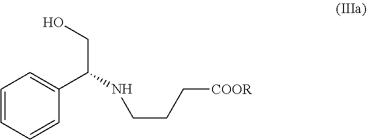

b) N-protection of compound of formula (IIIa) to obtain the compound of formula (III)

The eighth aspect of the present invention is depicted below scheme-IV.

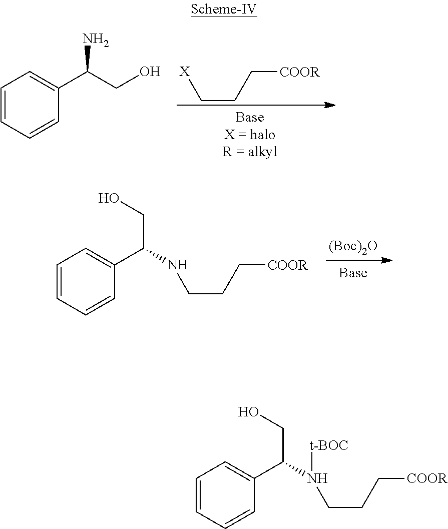

The term “halo” refers fluroro, chloro, bromo and iodo. The term alkyl refers methyl, ethyl, propyl, butyl, isopropyl and the like.

The reaction of R-(−)-2-phenylglycinol with 4-halao butyric acid alkyl ester carried in organic solvent. The organic solvent is selected from ether such as diethyl ether, 1,4-dioxane, tetrahydrofuran and the like; ketone such as acetone, methyl ethyl ketone and the like.

Base used in this reaction includes organic and inorganic base. Organic base is selected from amine such as methyl amine, ethyl amine, diethyl amine, triethyl amine, piperazine and the like. Inorganic base selected from alkali metal carbonates and hydoxides such as sodium carbonate, potassium carbonate, sodium hydroxide, potassium hydroxide and the like. The reaction is carried out in the temperature range of 0-100° C., preferably 20-90° C., more preferably 30-80° C.

N-protection of compound of formula (IIIa) is carried out using di-tert-butyl dicarbonate in organic solvent. The organic solvent is selected from ether such as diethyl ether, 1,4-dioxane, tetrahydrofuran and the like; ketone such as acetone, methyl ethyl ketone and the like.

Base used in this reaction includes organic and inorganic base. Organic base is selected from amine such as methyl amine, ethyl amine, diethyl amine, triethyl amine, piperazine and the like. Inorganic base selected from alkali metal carbonates and hydoxides such as sodium carbonate, potassium carbonate, sodium hydroxide, potassium hydroxide and the like. N-protection is carried out in the temperature range of −10 to 50° C., preferably 10 to 40° C., more preferably 20-35° C.

In each stage the compounds are isolated from the reaction mixture may involve methods including removal of solvent, cooling, crash cooling, concentrating the mass, evaporation, flash evaporation, simple evaporation, fast solvent evaporation, rotational drying, spray drying, thin-film drying, agitated thin film drying, agitated nutsche filter drying, pressure nutsche filter drying, freeze-drying, rotary vacuum paddle dryer, adding anti-solvent or the like. Stirring or other alternate methods such as shaking, agitation, or the like, may also be employed for the isolation.

The ninth aspect of the present invention provides a process for preparation of Elagolix or its pharmaceutically acceptable salt comprising:
a) converting the compound of formula (II) to compound of formula (VII) as described herein and
b) ester hydrolysis of compound of formula (VII) to Elagolix or its pharmaceutically acceptable salt.

The ester hydrolysis of compound of formula (VII) to Elagolix or its pharmaceutically acceptable salt is carried out by the process known in the literature.

### EXAMPLES

### Example 1: Preparation of Amorphous Elagolix Sodium

1 g of Elagolix sodium and 20 mL of methanol were charged into a round bottom flask at 30° C. The contents were stirred for dissolution and filtered. The filtrate was distilled at 65° C. to afford the title compound.

### Example 2: Preparation of Amorphous Elagolix Sodium

1 g of Elagolix sodium and 20 mL of methanol & Dichloromethane mixture (1:4) were charged into a round bottom flask at 28° C. The contents were stirred for dissolution and filtered. The filtrate was distilled at 45° C. to afford the title compound.

### Example 3: Preparation of Amorphous Elagolix Sodium

1.25 g of Elagolix sodium and 25 mL of Tetrahydrofuran were charged into a round bottom flask at 28° C. The contents were stirred for dissolution and filtered. The filtrate was distilled at 65° C. to afford the title compound.

### Example 4: Preparation of Amorphous Elagolix Sodium

1.0 g of Elagolix sodium was charged into a ball milling chamber at 28° C. and milled for about 2 hours to afford the title compound.

### Example 5: Preparation of Amorphous Elagolix Sodium

1.5 g of Elagolix sodium and 150 mL of N,N-Dimethyl Formamide were charged into a round bottom flask at 28° C. The contents were stirred for dissolution and filtered. The filtrate was spray dried at 130° C. to afford the title compound.

### Example 6: Preparation of Amorphous Solid Dispersion of Elagolix Sodium

0.2 g of Elagolix sodium and 0.2 g of Syloid® were added into Mortar and pestle. The contents were grinded for five minutes to get the title compound.

### Example 7: Preparation of Amorphous Solid Dispersion of Elagolix Sodium

0.5 g of Elagolix sodium and 0.5 g of Soluplus® were added to 20 mL of methanol&Dicholromethane mixture (1:4) at 28° C. The contents were stirred for dissolution and filtered. The filtrate was distilled under reduced pressure at 45° C. to afford the title compound.

### Example 8: Preparation of Amorphous Solid Dispersion of Elagolix Sodium

0.5 g of Elagolix sodium and 0.5 g of PEG-8000 were added to 20 mL of methanol & dichloromethane mixture (1:4) at 28° C. The contents were stirred for dissolution and filtered. The filtrate was distilled under reduced pressure at 45° C. to afford the title compound.

### Example 9: Preparation of Amorphous Solid Dispersion of Elagolix Sodium

0.25 g of solid dispersion of Elagolix sodium obtained from the above example 7 and 0.125 g of Neusilin® ULF2 were added into Mortar and pestle. The contents were grinded for ten minutes to get the title compound.

### Example 10: Preparation of Amorphous Solid Dispersion of Elagolix Sodium

0.25 g of Elagolix sodium and 0.25 g of Neusilin® were added into Mortar and pestle. The contents were grinded for five minutes to get the title compound.

### Example 11: Preparation of ethyl (R)-4-((2-hydroxy-1-phenylethyl)amino)butanoate (IIIa; R is ethyl)

R-(−)-2-phenylglycinol (10 g), DMAP (0.17 g) were added in THF (80 ml) at room temperature under nitrogen atmosphere. Triethylamine (30.48 ml) was added to the reaction mixture and stirred for five minutes. Ethyl-4-bromo butyrate (15.64 ml) was added and the reaction mixture heated to 80° C. then stirred for 16 hours. Water (20 volumes) followed by ethyl acetate (200 ml) were added to separate the aqueous and organic layer. The organic layer was washed with 1N HCl (100 ml) followed by neutralize the resulting aqueous layer with saturated sodium carbonate solution then extract with ethyl acetate (100 ml) and the organic layer was dried over anhydrous sodium sulfate then evaporated below 50° C. under reduced pressure to obtain the title compound. Yield: 14.50 g. Purity: 94.75% (by HPLC). ¹H NMR (400 MHz, DMSO-d₆): δ 7.17-7.30 (m, 5H), 4.83 (m, 1H), 3.99 (q, 2H), 3.58 (dd, 1H, J=8.8, 4.4 Hz), 3.88 (m, 1H), 3.27 (m, 1H), 2.38 (m, 1H), 2.26 (m, 3H), 2.10 (s, 1H), 1.61 (m, 2H), 1.12 (t, 3H); m/z: 252 (MH⁺)

### Example 12: Preparation of ethyl (R)-4-((tert-butoxycarbonyl)(2-hydroxy-1-phenylethyl) amino)butanoate (III; R is ethyl)

Ethyl (R)-4-((2-hydroxy-1-phenylethyl)amino)butanoate (14 g) was added to THF (140 ml) at room temperature. The reaction mixture was cooled to 0-5° C. Triethylamine (16.9 mL) was added to the reaction mixture followed by Di-tert-butyl dicarbonate (13.37 g) was added to reaction mixture at 0-5° C. The reaction mixture was heated to room temperature and stirred for 16 hours. Water (300 mL) and ethyl acetate (300 mL) were added and the layers were separated. The organic layer was washed with sodium chloride then died over sodium sulfate followed by evaporation at 45° C. to obtain the crude compound. The crude compound was purified by silica gel (60/120 mesh) with 15-20% EtOAc/Hexane to obtain the title compound as a pale yellow syrup. Yield: 9.5 g. Purity: 95.42% (by HPLC). ¹H NMR (400 MHz, CDCl₃): δ 7.24-7.34 (m, 5H), 5.08 (m, 1H), 4.09 (m, 4H), 3.10 (m, 2H), 3.00 (s, 1H), 2.21 (m, 2H), 1.82 (m, 2H), 1.46 (s, 9H), 1.23 (t, 3H). m/z: 352.20 (MH⁺)

### Example 13: Preparation of ethyl (R)-4-((2-(5-bromo)-3-(2-fluoro-6-trifluoromethyl)benzyl)-4-methyl-2,6-dioxo-3,6-dihydropyrimidin-1(2H)-yl)-1-phenylethyl)(tert-butoxycarbonyl) amino)butanoate (IV; R is ethyl)

Ethyl (R)-4-((tert-butoxycarbonyl)(2-hydroxy-1-phenylethyl) amino)butanoate (III; R is ethyl) (1.0 g), 5-bromo-1-(2-fluoro-6-trifluoromethyl)benzyl-6-methylpyrimidine-2,4 (1H, 3H)-dione (II) (1.08 g), Triphenyl phosphine (1.49 g) were added to THF (30 mL) at room temperature under nitrogen atmosphere. DIAD (1.11 mL) was added to the reaction mixture and stirred for 16 hours at room temperature. Water (60 volume) was added to the reaction mixture followed by ethylacetate (60 mL) was added then the layers were separated. The organic layer was dried over sodium sulfate and evaporated below 50° C. under reduced pressure to obtain the crude compound. The crude compound was purified by silica gel (60/120 mesh) with 15-20% EtOAc/Hexane to obtain the title compound. Yield (1.3 g). Purity: 68.87% (by HPLC); ¹H NMR (DMSO-d6) δ 1.15-2.0 (11H), 2.43-2.48 (4H), 3.9 (2H), 4.71-4.8 (5H), 5.3-5.4 (3H), 7.28-7.3 (8H), 8.4 (2H); m/z: 616 (M-BOC)⁺

### Example 14: Preparation of ethyl (R)-4-((tert-butoxycarbonyl)-2-(5-(2-fluoro-3-methoxyphenyl)-3-(2-fluoro-6-trifluoromethyl)benzyl)-4-methyl-2,6-dioxo-3,6-dihydropyrimidin-1(2H)-yl)-1-phenylethyl)amino)-butanoate (VI; R is ethyl)

Ethyl (R)-4-((2-(5-bromo)-3-(2-fluoro-6-trifluoromethyl)benzyl)-4-methyl-2,6-dioxo-3,6-dihydropyrimidin-1(2H)-yl)-1-phenylethyl)(tert-butoxycarbonyl) amino)butanoate (IV; R is ethyl) (0.9 g), 2-fluoro-3-methoxy phenyl boronic acid (V) (0.214 g) and sodium carbonate (0.797 g) were added to the mixture of 1,4-dioxane (9 mL) and water (3.06 mL) at room temperature under nitrogen atmosphere. Argon gas was bubbled through for 30 minutes. Tetrakis (triphenylphosphine)palladium (0.145 g) was added to the reaction mixture at room temperature then heated to 90-95° C. and stirred for 5 hours. The reaction mixture cooled to room temperature and filtered through celite bed then the filtrate washed with ethylacetate (9 mL) and water (36 mL) was added and stirred for 30 minutes at room temperature. Ethylacetate (36 mL) was added and the separated organic layer washed with brine and dried over sodium sulfate followed by evaporation at 45° C. to obtain the crude compound. The crude compound was purified by silica gel (60/120 mesh) with 20-25% EtOAc/Hexane to obtain the title compound as yellow solid. Yield: 0.5 g; Purity: 75.1% (by HPLC); m/z: 660 (M-BOC)⁺.

### Example 15: Preparation of ethyl (R)-4-((2-(5-(2-fluoro-3-methoxyphenyl)-3-(2-fluoro-6-trifluoromethyl)benzyl)-4-methyl-2,6-dioxo-3,6-dihydropyrimidin-1(2H)-yl)-1-phenylethyl)amino)-butanoate (VII; R is ethyl)

Ethyl(R)-4-((tert-butoxycarbonyl)-2-(5-(2-fluoro-3-methoxyphenyl)-3-(2-fluoro-6-trifluoro methyl)benzyl)-4-methyl-2,6-dioxo-3,6-dihydropyrimidin-1(2H)-yl)-1-phenylethyl)amino)-butanoate (VI; R is ethyl) (0.4 g) was added to dichloromethane (4 mL) at room temperature. The reaction mixture was cooled to 0-5° C. then trifluoroacetic acid (2 mL) was added and stirred for five hours at 0-5° C. Saturated sodium bicarbonate solution (40 mL) was added to the reaction mixture followed by dichloromethane (40 mL) was added. The organic layer was washed with brine then dried over sodium sulfate and evaporated at 35° C. to obtain the crude compound. The crude compound purified by silica gel (60/120 mesh) with 30-35% EtOAc/Hexane to obtain the title compound as yellow solid. Yield: 160 mg; Purity: 88.6% (by HPLC). ¹H NMR (400 MHz, DMSO-d₆): δ 7.64 (m, 1H), 7.54 (m, 2H), 7.15-7.27 (m, 6H), 6.85 (m, 2H), 5.31 (s, 2H), 3.99 (m, 3H), 3.87 (m, 2H), 3.83 (s, 3H), 2.30-2.16 (m, 4H), 2.10 (s, 3H), 1.50 (m, 2H), 1.10 (t, 3H). m/z: 660 (MH⁺)

1. A process for the preparation of amorphous Elagolix sodium, comprising the steps of:
a) providing a solution of Elagolix sodium in a solvent or mixture of solvents; and
b) isolating amorphous Elagolix sodium.

2. A process for the preparation of amorphous Elagolix sodium, comprising the step of:
a) ball milling Elagolix sodium under suitable milling conditions.

3. Amorphous solid dispersion of Elagolix sodium, wherein the Elagolix sodium is dispersed with one or more pharmaceutically acceptable carriers selected from Polyethylene glycol, Syloid®, Soluplus® and Neusilin®.

4. A process for the preparation of amorphous solid dispersion of Elagolix sodium comprising the steps of:
a) providing a solution comprising Elagolix sodium and one or more pharmaceutically acceptable carriers in a solvent,
b) removing the solvent from the solution obtained in step (a) and,
c) recovering amorphous solid dispersion of Elagolix sodium.

5. The process of claim 3, wherein the pharmaceutically acceptable carrier is selected from Polyethylene glycol, Syloid®, Soluplus® and Neusilin®.

6. A solid dispersion comprising amorphous Elagolix sodium and one or more pharmaceutically acceptable carriers wherein pharmaceutically acceptable carrier is selected from Polyethylene glycol, Syloid®, Soluplus® and Neusilin®.

7. A process for the preparation of solid dispersion comprising amorphous Elagolix sodium and one or more pharmaceutically acceptable carriers, comprising the steps of:
a) providing a solution comprising Elagolix sodium and one or more pharmaceutically acceptable carriers in a solvent;
b) removing the solvent from the solution obtained in step (a); and
c) recovering a solid dispersion comprising amorphous Elagolix sodium and one or more pharmaceutically acceptable carriers.

8. The process of claim 5, wherein the pharmaceutically acceptable carrier is selected from Polyethylene glycol, Syloid®, Soluplus® and Neusilin®.

9. A process for preparation of solid dispersion comprising amorphous Elagolix sodium and one or more pharmaceutically acceptable carriers, comprising the steps of:
a) grinding Elagolix sodium and one or more pharmaceutically acceptable carriers and;
b) recovering a solid dispersion comprising amorphous Elagolix sodium and one or more pharmaceutically acceptable carriers.

10. The process of claim 7, wherein the pharmaceutically acceptable carrier is selected from Polyethylene glycol, Syloid®, Soluplus® and Neusilin®.

11. A pharmaceutical composition comprising amorphous Elagolix sodium.

12. A pharmaceutical composition comprising amorphous solid dispersion of Elagolix sodium.

13. A solid dispersion pharmaceutical composition comprising solid dispersed Elagolix sodium.

14. A method for preparing a pharmaceutical composition, the method comprising combining the Elagolix sodium of claim 1 and at least one pharmaceutically acceptable excipient.

15. A process for preparation of compound of formula (VII) 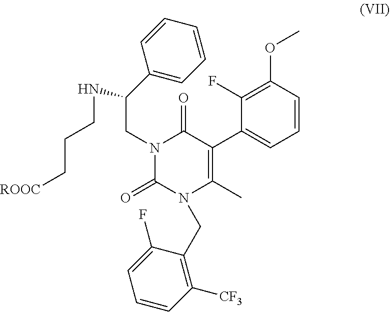
wherein R is alkyl such as methyl, ethyl, propyl, isopropyl and the like, comprising:
a) reacting the compound of formula (II) with compound of formula (III) to obtain the compound of formula (IV) 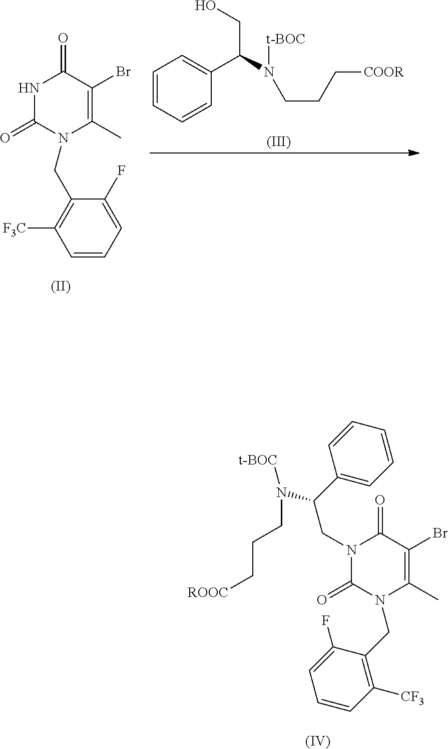
wherein t-BOC is tertiary butoxycarbonyl group; R is as described above;
b) reacting the compound of formula (IV) with the compound of formula (V) to obtain the compound formula (VI), and 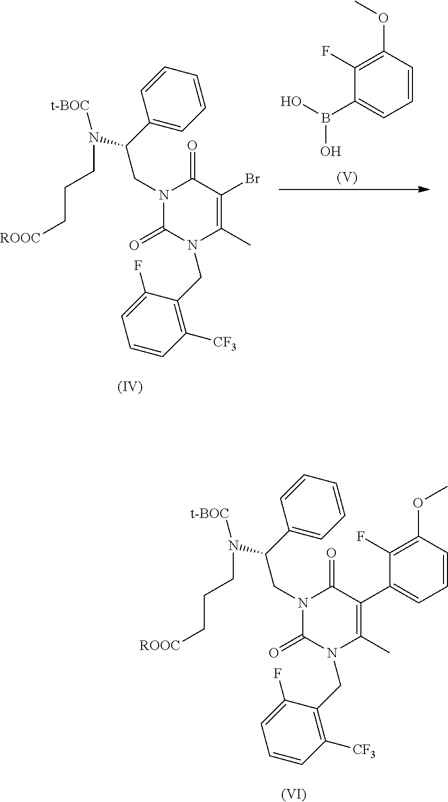
c) N-deprotection of the compound of formula (VI) to obtain the compound of formula (VII)

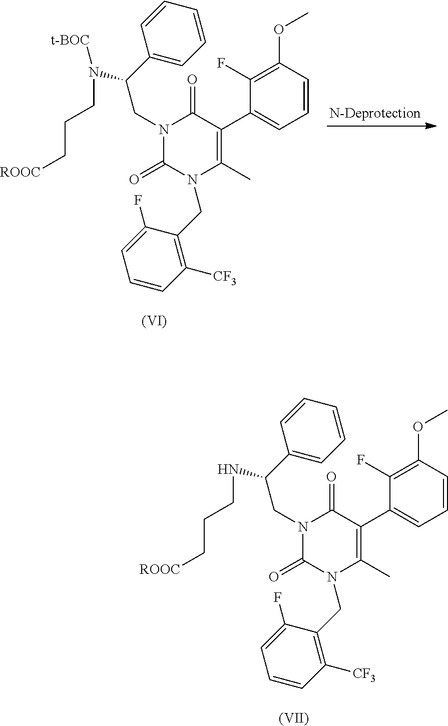

16. The process of claim 11, wherein the reaction of compound of formula (II) with compound of formula (III) to obtain the compound of formula (IV) is carried in the presence of triarylphosphine and azodicarboxylates.

17. A process for the preparation of compound of formula (III) 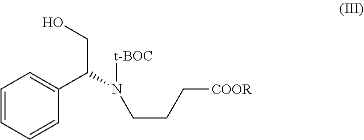
wherein R is alkyl such as methyl, ethyl, propyl and the like, comprising:
a) reacting R-(−)-2-phenylglycinol with 4-halao butyric acid alkyl ester to obtain the compound of formula (IIIa) and 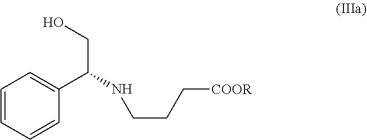
b) N-protection of compound of formula (IIIa) to obtain the compound of formula (III).

18. A process for preparation of Elagolix or its pharmaceutically acceptable salt comprising the steps of:
a) converting the compound of formula (II) to compound of formula (VII) as described herein and
b) ester hydrolysis of compound of formula (VII) to Elagolix or its pharmaceutically acceptable salt.
